(19) United States
(12) Patent Application Publication
(10) Pub. No.: US 2026/0034189 A1
(43) Pub. Date: Feb. 5, 2026

(54) All Natural Motion Sickness Solution

(71) Applicant: Dennis Brown, Rockwell, NC (US)

(72) Inventor: Dennis Brown, Rockwell, NC (US)

(21) Appl. No.: 18/831,128

(22) Filed: Aug. 2, 2024

(51) Int. Cl. A61K 36/906; A61K 36/28; A61K 36/534; A61K 45/06; A61P 1/08

(52) U.S. Cl. CPC A61K 36/906; A61K 36/28; A61K 36/534; A61K 45/06; A61P 1/08

(57) ABSTRACT

The present invention provides a natural, non-alcoholic ear drop formulation for treating motion sickness disorders, including vertigo and equilibrium disorders. The composition includes only natural, unadulterated or minimally processed oils, ensuring efficacy and safety without side effects. The targeted ear drop application provides effective relief by lubricating the inner ear structures responsible for balance and motion sensing.

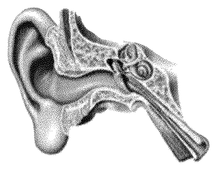

Only use this product only when you feel that motion sickness will become a problem. Repeat only when necessary.

As a disclaimer this product is just a remedy and not a cure for motion sickness. Results may vary between each individual.

Always consider talking with your medical provider before taking this product.

A.N.M.S.S Technical Disclosure the Automated Network Monitoring and Security System (A.N.M.S.S) enhance network security and performance through continuous, real-time monitoring and advanced threat detection. By leveraging machine learning and data analysis, A.N.M.S.S identifies anomalies and automatically responds to potential security threats. Key features include real-time traffic monitoring, machine learning-based anomaly detection, automated threat response, detailed reporting, and scalability for various network sizes. A.N.M.S.S is designed for enterprise networks, data centers, and Iota networks, ensuring optimal functionality and robust protection of digital assets.

The formulation is a non-alcoholic composition for treating the effects of motion sickness disorders, including vertigo, equilibrium disorders. The formulation includes only natural ingredients. All oils in the formulation are unadulterated or minimally processed and do not result in irritation to the skin or other harmful side effects. The composition was designed as (ear drops) formulated as lubrication for the inner ear.

### DETAILED DESCRIPTION OF THE INVENTION

Composition: The formulation of the All Natural Motion Sickness Solution could includes but not necessary the following natural ingredients, each selected for their efficacy and safety: Preparation Method:
1. Selection of Ingredients: All oils are selected based on their purity, with preference given to unadulterated or minimally processed oils to ensure safety and effectiveness.
2. Mixing: The oils are combined in specific proportions to create a balanced formulation that maximizes the therapeutic effects while minimizing any potential side effects.
3. Bottling: The mixture is then placed in sterile, dropper-equipped bottles to allow for precise application to the inner ear.

Application:

The composition is designed to be used as ear drops. 1st clean any excessive wax from the ear, gently squeeze the bottle until the oil enters the ear canal, apply a piece of cotton ball to keep the oil from leaking out. Repeat the process for the other side of the ear. The formulation provides lubrication and targets the inner ear structures responsible for balance and motion sensing. This targeted approach helps alleviate symptoms of motion sickness more effectively than systemic medications.

### BACKGROUND OF THE INVENTION

1. Field of the Invention The present invention relates, to a composition for treating motion sickness disorders. The composition was designed as (ear drops) formulated as lubrication for the inner ear composition of natural ingredients for relieving the effects of motion in the inner ear.

2. Description of the Related Problem The ear is made up of the outer ear, middle ear, and inner ear. The inner ear consists of the bony labyrinth and membranous labyrinth. The bony labyrinth comprises three components:

### BRIEF DESCRIPTION OF THE DRAWINGS

The ear is made up of the outer ear, middle ear, and inner ear. The inner ear consists of the bony labyrinth and membranous labyrinth. The bony labyrinth comprises three components:
Cochlea;

The cochlea is made of a hollow bone shaped like a snail and divided into two chambers by a membrane. The chambers are full of fluid, which vibrates when sound comes in and causes the 30,000 tiny hairs1 lining the membrane to vibrate and send electrical impulses (sound signals) to the brain. The cochlea is about 9 millimeters wide at its widest point, and about 5 millimeters tall. If it could be uncoiled, the cochlea would be about 30 millimeters long. Semicircular Canals;

Also known as the labyrinthine, the semicircular canals rest on top of the cochlea, connected by the vestibule. There are three of them, and they line up at 90-degree angles to one another, which allow the brain to know which direction the head is moving. Like the cochlea, these canals are filled with fluid. They also contain small calcium crystals and tiny hairs that sense the movement of the fluid. The size of the canals is difficult to measure since they are surrounded by bone3
Vestibule:

The vestibule is the central part of the bony labyrinth. It is separated from the middle ear by the oval window, and communicates anteriorly with the cochlea and posteriorly with the semicircular canal. Inside the bony labyrinth lies the membranous labyrinth, which is also made up of three parts:
Cochlear duct: This triangle-shaped duct is located inside the bony labyrinth and creates two canals that sit above and below it. These two canals—the scala vestibuli above the duct and the scala tympani below it—are separated from the main duct by membranes. The membrane between the cochlear duct and the scala tympani—also known as the basilar membrane—is where the primary hearing organ, the Organ of Corti, is located. The upper membrane is called Reissner's membrane, which helps control the flow of fluid from the duct to the scala vestibule
Semicircular ducts: This is where fluid, called endolymph, changes speed and direction when you move your head. Sensory receptors in these ducts detect this change and send information to your brain to help you maintain balance
Utricle and saccule: These sacs are within the membranous labyrinth and work to detect vertical and horizontal positioning. Endolymph enters and exits these sacs The final structural pieces of the inner ear are the seventh and eighth cranial nerves, which carry information about sound and balance to the brain:
Cranial Nerve 1:

Also called the facial nerve, this nerve provides information to the brain on sensory and motor function. One section of the nerve controls the motor function of some part of the face, and another controls sensory information related to taste and hearing. Specifically, this nerve carries information to the brain from the tympanic membrane.
Cranial Nerve 2:

Another cranial nerve, the eighth cranial nerve is also called the vestibule ocochlear nerve. It combines vestibular and cochlear nerve fibers. The vestibular nerve fibers provide information to the brain about balance, while the cochlear nerve fibers carry information about sound. Both are sensory nerve fibers. Damage to this nerve could impact both hearing and balance
Motion Sickness,

As the term is used herein, encompasses numerous movement conditions, disorders ranging from
Dizziness
Vertigo Motion sickness
Nausea or vomiting
Unsteadiness or frequent falls
Lightheadedness
Headaches
Video head impulse accrues when goggles are placed on your face, and you track targets with your eyes. An audiologist will then produce certain movements to stimulate different parts of the inner ear, to the different movements. Until now, the treatment of motion sickness disorders has been largely based on non-specific drugs, and only limited success has been achieved. What is motion sickness?Motion sickness occurs when your brain can't make sense of information sent from your eyes, ears and body. Lots of motion—in a car, airplane, boat, or even an amusement park ride—can make you feel queasy, clammy or sick to your stomach. Some people vomit.

Being carsick, seasick or airsick is motion sickness. Who might get motion sickness an estimated one in three people get motion sickness at some point?Women and children age two to 12 are most at risk. Still, the condition can affect anyone. What causes motion sickness? Your brain receives signals from motion-sensing parts of your body: your eyes, inner ears, muscles and joints. When these parts send conflicting information, your brain doesn't know whether you're stationary or moving. Your brain's confused reaction makes you feel sick. For example, when riding in a car, your:
Eyes see trees passing by and register movement.
Inner ears sense movement.
Muscles and joints sense that your body is sitting still.
Brain senses and disconnect among these messages.
Many actions can trigger motion sickness, such as:
Amusement park rides and virtual reality experiences.
Reading while in motion.
Riding in a boat, car, bus, train or plane.
Video games and movies. What are the symptoms of motion sickness?Motion sickness can take you by surprise. You may feel fine one moment and then suddenly experience some of these symptoms:
Cold sweats.
Dizziness.
Fatigue.
Headache.
Irritability.
Inability to concentrate.
Increased saliva, nausea and vomiting.
Pale skin.
Rapid breathing or gulping for air. How is motion sickness managed or treated? You have some options to prevent motion sickness or treat symptoms. Motion sickness treatments include:
Antihistamines: Commonly used to treat allergies, antihistamines can also prevent motion sickness and ease symptoms. Only antihistamines that cause drowsiness are effective. Non drowsy formulas won't help.
Patches: Scopolamine skin patches or oral pills prevent nausea and vomiting. You stick the patch behind your ear at least four hours before traveling. After three days, you remove the patch and apply a new one. This medication can cause dry mouth and is only approved for adults. What are the complications of motion sickness?Motion sickness doesn't tend to cause serious problems. Some people can't stop throwing up. Excessive vomiting can cause dehydration and low blood pressure (hypotension)

### SUMMARY OF THE INVENTION

The present invention relates to a composition for treating motion sickness disorders, and particularly to a non-alcoholic composition of natural Ingredients for relieving the effects of motion in the inner ear. The topical composition includes several herbs and vitamins, including grape seed extract and vitamin E, and in one embodiment, a few drops of Essential Oil. Olive oil, Almond oil, Vitamin d, apricot oil, jojoba oil, grape seed oil, primrose oil, Vitamin E oil, carrot seed oil, and rosemary oil. None of the above essential oils, taken either singly or in combination, is seen to describe the instant invention as claimed. In particular, none of the above essential oils has described a composition combining all of the naturally occurring ingredients of the present motion sickness formulation application, thus a formulation of some or all of the oils proves effective in solving the aforementioned problems as desired. The essential oil formulation of the present invention is a non-alcoholic composition for treating and alleviating motion sickness. The formulation includes only natural ingredients. All essential oils in the formulation are unadulterated or minimally processed and do not result in irritation to the skin or other harmful side effects. The compositions can be formulated as a tropical ointment for lubrication ear drops of the inner ear canal. Accordingly, it is a principal object of the invention to provide a natural motion sickness remedy that could relive discomforts from motion, vertigo, and equilibrium disorders. It is another object of the invention to provide a natural motion sickness remedy which includes only ingredients that are from a natural source. It is a further object of the invention to provide a motion sickness remedy which is alcohol free. Still another object of the invention is to provide a motion sickness remedy which does not cause adverse side effects. It is an object of the invention to provide improved elements and arrangements thereof for the purposes described which is inexpensive, the formulation is a non-alcoholic composition for treating the effects of motion sickness disorders, including vertigo, equilibrium disorders dependable and fully effective in accomplishing its intended purposes. These and other objects of the present invention will become readily apparent upon further review of the following specification.

### DETAILED DESCRIPTION OF THE PREFERRED EMBODIMENTS EXAMPLE

EXAMPLE 1 essential oils are compounds extracted from plants. The oils capture the plant's scent and flavor, or “essence.” Unique aromatic compounds give each essential oil its characteristic essence. Essential oils are obtained through distillation (via steam and/or water) or mechanical methods, such as cold pressing. Once the aromatic chemicals have been extracted, they are combined with a carrier oil to create a product that's ready for use. The way the oils are made is important, as essential oils obtained through chemical processes are not considered true essential oils. The formulation is a non-alcoholic composition for treating the effects of motion sickness disorders, including vertigo, equilibrium disorders. Essential oils are concentrated plant extracts that retain the natural “essence,” of their source.

EXAMPLE 2 Essential oils are most commonly used in the practice of aromatherapy, in which they are inhaled through various methods. Essential oils are not meant to be swallowed. The chemicals in essential oils can interact with your body in several ways. When applied to your skin, some plant chemicals are absorbed (1Trusted Source, 2Trusted Source). It's thought that certain application methods can improve absorption, such as applying with heat or to different areas of the body. The formulation is a non-alcoholic composition for treating the effects of motion sickness disorders, including vertigo, equilibrium disorders. However, research in this area is lacking.

However, this has yet to be confirmed in studies.

### SUMMARY

Essential oils can be inhaled or diluted and applied to the skin. They may stimulate your sense and have medicinal effects when absorbed.

EXAMPLE 3 Here's a list of 10 popular essential oils and the health claims associated with them:
Peppermint: used to boost energy and aid digestion
Lavender: used to relieve stress
Sandalwood: used to calm nerves and help with focus
Bergamot: used to reduce stress and improve skin conditions like eczema
Rose: used to improve mood and reduce anxiety
Chamomile: used to improve mood and relaxation
Ylang-Ylang: used to treat headaches, nausea, and skin conditions
Tea Tree: used to fight infections and boost immunity
Jasmine: used to help with depression, childbirth, and libido
Lemon: used to aid digestion, mood, headaches, and more

### SUMMARY

There are over 90 commonly used essential oils, each associated with certain health claims. Popular oils include peppermint, lavender, and sandalwood. The formulation is a non-alcoholic composition for treating the effects of motion sickness disorders, including vertigo, equilibrium disorders.

### EXAMPLE

Stress and anxiety It has been estimated that 43% of people who have stress and anxiety use some form of alternative therapy to help relieve their symptoms. Regarding aromatherapy, initial studies have been quite positive. Many have shown that the smell of some essential oils can work alongside traditional therapy to treat anxiety and stress.

However, due to the scents of the compounds, it's hard to conduct blinded studies and rule out biases. The formulation is a non-alcoholic composition for treating the effects of motion sickness disorders, including vertigo, equilibrium disorders. Thus, many reviews on the stress- and anxiety-relieving effects of essential oils have been inconclusive.

Interestingly, using essential oils during a massage may help relieve stress, although the effects may only last while the massage is taking place.

A recent review of over 201 studies found that only 10 were robust enough to analyze. It also concluded that aromatherapy was ineffective at treating anxiety.

### EXAMPLE

It has been suggested that essential oils may help fight inflammatory conditions. Some test-tube studies show that they have anti-inflammatory effects.

One mouse study found that ingesting a combination of thyme and oregano essential oils helped induce the remission of colitis. Two rat studies on caraway and rosemary oils found similar results.

However, very few human studies have examined the effects of these oils on inflammatory diseases. Therefore, their effectiveness and safety are unknown.

Antibiotic and antimicrobial the rise of antibiotic-resistant bacteria has renewed interest in the search for other compounds that can fight bacterial infections. Test-tube studies have investigated essential oils, such as peppermint and tea tree oil, extensively for their antimicrobial effects, observing some positive results.

However, while these test-tube study results are interesting, they do not necessarily reflect the effects that these oils have within your body. The formulation is a non-alcoholic composition for treating the effects of motion sickness disorders, including vertigo, equilibrium disorders. They don't prove that a particular essential oil could treat bacterial infections in humans.

### SUMMARY

Essential oils may have some interesting health applications. However, more research is needed in humans. How to choose the right essential oils many companies claim that their oils are “pure” or “medical grade.” However, these terms aren't universally defined and therefore hold little weight. Given that they're products of an unregulated industry, the quality and composition of essential oils can vary greatly.

Keep the following tips in mind to choose only high-quality oils:
Purity: Find oil that contains only aromatic plant compounds, without additives or synthetic oils. Pure oils usually list the plant's botanical name (such as Lavandula officinalis) rather than terms like “essential oil of lavender.”
Quality: True essential oils are the ones that have been changed the least by the extraction process. Choose a chemical-free essential oil that has been extracted through distillation or mechanical cold pressing.
Reputation: Purchase a brand with a reputation for producing high-quality products.

### SUMMARY

High-quality oils only use pure plant compounds extracted by distillation or cold pressing. Avoid oils that have been diluted with synthetic fragrances, chemicals, or oils. Safety and side effects just because something is natural doesn't mean its safe. The formulation is a non-alcoholic composition for treating the effects of motion sickness disorders, including vertigo, equilibrium disorders. Plants and herbal products contain many bioactive compounds that may harm your health, and essential oils are no different. However, when inhaled or combined with base oil for use on your skin, most essential oils are considered safe. Be sure to consider others in your environment who might be inhaling the aroma, including pregnant women, children, and pets.

Nevertheless, they may cause some side effects, including:
Rashes
Asthma attacks
Headaches
allergic reactions While the most common side effect is a rash, essential oils can cause more serious reactions, and they have been associated with one case of death.

The oils that have most commonly been associated with adverse reactions are lavender, peppermint, tea tree, and ylang-ylang. Oils that are high in phenols, such as cinnamon, can cause skin irritation and shouldn't be used on the skin without being combined with a base oil.

Meanwhile, essential oils made from citrus fruits increase the skin's reaction to sunlight and burns can occur. Swallowing essential oils is not recommended, as doing so could be harmful and, in some doses, fatal.

Very few studies have examined the safety of these oils for pregnant or breastfeeding women, who are usually advised to avoid them.

Only use this product only when you feel that motion sickness will become a problem.

Repeat only when necessary.

As a disclaimer this product is just a remedy and not a cure for motion sickness.

Results may vary between each individual.

Always consider talking with your medical provider before taking this product.

A.N.M.S.S Technical Disclosure the Automated Network Monitoring and Security System (A.N.M.S.S) enhance network security and performance through continuous, real-time monitoring and advanced threat detection. By leveraging machine learning and data analysis, A.N.M.S.S identifies anomalies and automatically responds to potential security threats. Key features include real-time traffic monitoring, machine learning-based anomaly detection, automated threat response, detailed reporting, and scalability for various network sizes.

A.N.M.S.S is designed for enterprise networks, data centers, and Iota networks, ensuring optimal functionality and robust protection of digital assets.

### All Natural Motion Sickness Solution Inc.

238 BIRTWICK RD ROCKWELL, NC 28138 Web. https://www.naturalmotionsicknesssolution.com/Email. dennisbrown@naturalmotionsicknesssolution.com 1-828-390-6097

### Non provisional utility patent

### All Natural Motion Sickness Solution

### DETAILED DESCRIPTION OF THE INVENTION

### Composition:

The formulation of the All Natural Motion Sickness Solution includes natural ingredients selected for their efficacy and safety. All oils in the formulation are unadulterated or minimally processed, ensuring they do not irritate the skin or cause other harmful side effects. The composition is designed as ear drops, formulated to lubricate the inner ear.

### Preparation Method:

1. Selection of Ingredients:
   All oils are selected based on their purity, with preference given to unadulterated or minimally processed oils to ensure safety and effectiveness.
2. Mixing:
   The oils are combined and heated in specific proportions to sterilize create a balanced formulation that maximizes therapeutic effects while minimizing any potential side effects.
3. Bottling:
   The mixture is then placed in sterile, dropper-equipped bottles to allow for precise application to the inner ear.

### Application:

The composition is designed to be used as ear drops. Clean any excess wax from the ear, gently squeeze the bottle until the oil enters the ear canal, and apply a piece of cotton ball to keep the oil from leaking out. Repeat the process for the other ear. The formulation provides lubrication and targets the inner ear structures responsible for balance and motion sensing, alleviating symptoms of motion sickness more effectively than systemic medications.

### BACKGROUND OF THE INVENTION

### 1. Field of the Invention

The present invention relates to a composition for treating motion sickness disorders. The composition is designed as ear drops formulated to lubricate the inner ear with natural ingredients to relieve the effects of motion in the inner ear.

### 2. Description of the Related Problem

The ear is made up of the outer ear, middle ear, and inner ear. The inner ear consists of the bony labyrinth and membranous labyrinth. The bony labyrinth comprises three components:
Cochlea: A hollow bone shaped like a snail, divided into two chambers by a membrane. The chambers are filled with fluid, which vibrates when sound comes in, causing the tiny hairs lining the membrane to vibrate and send electrical impulses (sound signals) to the brain.
Semicircular Canals: These canals rest on top of the cochlea and are connected by the vestibule. There are three canals that line up at 90-degree angles to one another, allowing the brain to detect head movement direction.
Vestibule: The central part of the bony labyrinth, separated from the middle ear by the oval window, and communicating with the cochlea and semicircular canals.

Inside the bony labyrinth lies the membranous labyrinth, which includes:
Cochlear Duct: Located inside the bony labyrinth, creating two canals above and below it. The primary hearing organ, the Organ of Corti, is located here.
Semicircular Ducts: These ducts contain fluid that changes speed and direction with head movement, helping maintain balance.
Utricle and Saccule: Sacs that detect vertical and horizontal positioning.

The final structural pieces of the inner ear are the seventh and eighth cranial nerves, carrying information about sound and balance to the brain.

### Motion Sickness:

Motion sickness encompasses numerous movement conditions and disorders, including dizziness, vertigo, nausea, vomiting, unsteadiness, and headaches. It occurs when the brain cannot make sense of information sent from the eyes, ears, and body. Common triggers include car rides, boat trips, airplane flights, amusement park rides, and virtual reality experiences.

### SUMMARY OF THE INVENTION

The present invention relates to a non-alcoholic composition of natural ingredients for relieving motion sickness disorders by targeting the inner ear. The topical composition includes herbs and vitamins, such as grape seed extract and vitamin E, and essential oils. The formulation provides effective relief from motion sickness without adverse side effects.

### DETAILED DESCRIPTION OF THE PREFERRED EMBODIMENTS

### Example 1

Essential oils are compounds extracted from plants, capturing the plant's scent and flavor. The formulation is a non-alcoholic composition for treating the effects of motion sickness disorders, including vertigo and equilibrium disorders.

### Example 2

Essential oils are most commonly used in aromatherapy. When applied to the skin, some plant chemicals are absorbed, providing medicinal effects. The formulation is a non-alcoholic composition for treating motion sickness disorders.

### Example 3

A list of popular essential oils and their health claims includes peppermint (boosts energy, aids digestion), lavender (relieves stress), sandalwood (calms nerves), bergamot (reduces stress), and chamomile (improves mood).

### Example 4

Studies suggest that essential oils can help relieve stress and anxiety. The formulation is a non-alcoholic composition for treating motion sickness disorders.

### Example 5

Essential oils may have anti-inflammatory and antimicrobial effects. The formulation is a non-alcoholic composition for treating motion sickness disorders.

### SUMMARY

Essential oils are generally safe when applied to the skin or inhaled. They provide complementary therapy for minor health problems and help relieve the symptoms of motion sickness.

### Example 6

The invention uses oils to help alleviate motion sickness. Oils include coconut oil, jojoba oil, apricot kernel oil, sweet almond oil, olive oil, argan oil, rosehip oil, black seed oil, grape seed oil, and avocado oil. These oils act as carriers for essential oils and provide therapeutic benefits.

1. A.N.M.S.S Composition: An all-natural motion sickness solution comprising of all natural oils similar to olive oil.

2. Method for Alleviating Motion Sickness: A method for preventing and alleviating symptoms of motion sickness. o Administering an effective amount of the all-natural motion sickness solution to an individual inner ear to help relieve the effects and symptoms of motion sickness. o Administering the solution in the form of an oil, before the onset of travel or at the first sign of motion sickness symptoms. The invention claimed is thus an all-natural motion sickness solution that balance your equilibrium to effectively prevent and alleviate motion sickness symptoms without causing adverse side effects The bottom line Essential oils are generally considered safe to apply to the skin if they've been combined with base oil. The formulation is a non-alcoholic composition for treating the effects of motion sickness disorders, including vertigo, equilibrium disorders. However, evidence supporting many of their associated health claims is lacking, and their effectiveness is often exaggerated. For minor health problems, using essential oils as a complementary therapy is likely harmless. However, if you have a serious health condition or are taking medication, you should discuss their use with your healthcare practitioner.
EXAMPLE This invention is based on the use of oils for the benefits of helping persons that may suffer from motion sickness, by listing the types of oils this would act as a deterrent for product claiming the use of any product that uses any oils for motion sickness.

1. Coconut oil Coconut oil is edible oil made from the meat of mature coconuts. It's available in refined or unrefined varieties. Unrefined coconut oil comes from fresh coconut meat. It's not processed with chemicals and retains its coconut aroma and flavor. Refined coconut oil comes from dried coconut meat, also called copra. It's bleached and deodorized to remove contaminants, as well as the distinct coconut aroma and flavor. Refined coconut isn't all-natural and isn't recommended for use as a carrier oil. Uses: Coconut oil contains skin-nourishing fatty acids and polyphenols, which make it great carrier oil for massage oils and skin care preparations.

2. Jojoba oil Jojoba oil comes from the seeds of the jojoba plant. It has a delicate, nutty aroma. Technically, jojoba isn't oil, but a wax with powerful moisturizing properties. It's thought to closely mimic sebum, the skin's natural oil. Using jojoba oil may help reduce the skin's oil production in acne-prone people by making the skin think it's produced enough oil. Uses: Jojoba oil absorbs easily in the skin and doesn't clog pores. This makes it a good carrier oil option for massage oils, facial moisturizers, and bath oils.

3. Apricot kernel oil Apricot kernel oil is made from apricot seeds, also known as kernels. It's emollient oil high in fatty acids and vitamin E. It absorbs easily into the skin and has a slightly sweet, nutty scent. You can buy edible apricot kernel oil or apricot kernel oil for cosmetic use only. Uses: Apricot kernel oil is thought to help soften and calm irritated, itchy skin. Use it as a carrier oil to make massage oils, bath oil, and hair care preparations.

4. Sweet almond oil Sweet almond oil has a strong, nutty aroma. Its edible oil made from the kernels of sweet almonds. The oil is lightweight and absorbs easily, and is a great moisturizer for dry skin. It's also used in general aromatherapy, but its strong scent may mask an essential oil's aroma. Uses: Sweet almond oil is one of the most popular carrier oils for skin care. It's great in massage oils, bath oils, and soaps.

5. Olive oil Olive oil comes from pressed olives. Its best known as a healthy, edible oil with a fruity aroma, but it's also used in aromatherapy as a carrier oil. Extra-virgin olive oil is the preferred variety for aromatherapy and skin care preparations. Olive oil's scent may interfere with the scent of some essential oils. Uses: It's packed with fatty acids and plant sterols, which make it great for cleansing and moisturizing dry skin. Use olive oil as carrier oil for massage, facial cleansers, hair care, and homemade soaps.

6. Argan oil Argan oil is made from kernels found inside the fruit of argan trees, which are native to Morocco. The oil is edible and is traditionally used to nourish the body inside and out. It has a nutty aroma and is rich in vitamins A and E, and monounsaturated fatty acids. Uses: Argan oil can help treat dry skin and hair, wrinkles, and skin inflammation. This makes it terrific carrier oil for general skin care and massage oils.

7. Rosehip oil Rosehips are the seeds of the Rosa rubiginosa bush or the Rosa moschata bush. The flowers of both bushes look different from traditional roses. When these flowers die and drop their petals, the rosehip is left behind. Rosehip oil is pressed from rosehips. Rosehip oil doesn't smell like a rose, though. It has a nutty, earthy scent. Uses: Rosehip oil is high in vitamins A and C. Vitamin A is a natural retinoid that helps fight aging, and both vitamins can help reverse the effects of the sun on your skin. Use it as carrier oil for, dry skin remedies, massage oils, and moisturizers.

8. Black seed oil Black seed oil is made from the Nigella sativa plant. Although it's lesser known than other carrier oils, its rich trusted source with unsaturated and saturated fatty acids. It's also thought to have anti-inflammatory abilities.

9. Grape seed oil Grape seed oil comes from grape-seeds. It's a byproduct of the winemaking process. It's rich in vitamin E, a nutrient thought to heal the skin and reduce wrinkles, although scientific research is inconsistent. Uses: Grape seed oil is lightweight, easily absorbed by the skin, and has a neutral scent. It's a good carrier oil to use with essential oils to make body oils and massage oils.

10. Avocado oil Avocado oil is heavy, thick, edible oil made from avocado fruit. It has a nutty aroma. Avocado oil is high in oleic acid, a monounsaturated fatty acid thought to help dry, damaged skin. Uses: This can be good carrier oil for dry skin remedies and body creams unless you're dealing with acne. Avocado oil may increase sebum production, so if your skin is acne-prone, check with your dermatologist before use.

11. Sunflower oil Sunflower oil is edible oil extracted from sunflower seeds. It has a neutral odor. The oil is said to act as a skin barrier against toxins and germs that cause infection, making it a great choice for irritated skin. Uses: It's thought to help soften skin, moisturize skin, and soothe irritation, so add this carrier oil to your massage oils or use for general skin care. Michelle Pugle is a Canadian-based health & wellness writer. She has a diploma in holistic nutritional therapy, a double bachelor's in English and Sociology, and a master's in research theories. Her work has been featured in magazines, anthologies, and on websites around the world. Evidence Based This article is based on scientific evidence, written by experts and fact checked by experts.
